(19) United States
(12) Patent Application Publication
(10) Pub. No.: US 2021/0236075 A1
(43) Pub. Date: Aug. 5, 2021

(54) RADIOLOGICAL IMAGING SYSTEM COMPRISING A PORTABLE X-RAY IMAGE DETECTOR AND AN ANTIDIFFUSION GRID

(71) Applicant: TRIXELL, Moirans (FR)

(72) Inventors: Farid BETRAOUI, MOIRANS (FR); Nicolas GERIN, MOIRANS (FR)

(21) Appl. No.: 17/152,587

(22) Filed: Jan. 19, 2021

(30) Foreign Application Priority Data
Jan. 31, 2020 (FR) 2000954

(51) Int. Cl. A61B 6/00

(52) U.S. Cl. CPC A61B 6/4291; A61B 6/4208; A61B 6/56; A61B 6/46; A61B 6/54

(57) ABSTRACT

A radiological imaging system includes a portable X-ray image detector and an antidiffusion grid comprising a support for receiving the portable detector, wherein: the portable detector is provided with a near distance radio communication module; and the antidiffusion grid comprises a near distance radio communication tag, provided with a memory of at least 10 kilobytes comprising data representative of the antidiffusion grid, disposed in such a way as to be capable of communicating with the near distance radio communication module when the detector is fully inserted in the receiving support; the portable detector being configured for determining locally if the antidiffusion grid and the portable detector are compatible or incompatible from read data representative of the antidiffusion grid supplied to the near distance radio communication module by the tag.

### CROSS-REFERENCE TO RELATED APPLICATIONS

This application claims priority to foreign French patent application No. FR 2000954, filed on Jan. 31, 2020, the disclosure of which is incorporated by reference in its entirety.

### FIELD OF THE INVENTION

The invention relates to a portable X-ray image detector capable of automatically and autonomously detecting and identifying an antidiffusion grid. The antidiffusion grid is a tool used in radiography for improving the quality of the radiological image by reducing the contribution of the diffused radiation, this grid is placed between the patient and the detector.

### BACKGROUND

The document CN103961116B (Siemens) is known, which discloses an antidiffusion grid identification system comprising an identification device of the bar code type, a bar code reader, and a remote control centre connected to a network. The bar code reader is used for identifying and transmitting the identification of the grid to the remote control centre, this control centre associates the antidiffusion grid data with data relative to the patient in order to verify if the association is correct or not and in order to record data relative to high voltage generator and to the X-ray tube.

Such a system is complex, expensive and has a certain latency because it has to call upon a remote control centre. It lengthens the sequence of operations carried out by the radiographer by requiring him to carry out the reading of the bar code of the antidiffusion grid before the acquisition of the image; in fact, in order for the antidiffusion grid to function, it must be in contact with the detector whereas in order for the reading of the bar code to be carried out a distance of several centimetres is necessary. There is no integrated device for warning the user in the case of inadequacy between the antidiffusion grid and the detector.

The document U.S. Pat. No. 8,634,517 (Philips) is also known, which describes a portable radiographic detector with an antidiffusion grid capable associating the detector and the radiographic system in order to determine the adjustments required for an automatic exposure by a portable detector on the basis of data exchanged with the detector. It mentions the use of the RFID technology for establishing the communication between the grid support and the X-ray detector. The detector is passive and all of the data is managed by a remote control centre outside of the detector-grid assembly.

Such an arrangement is complex, expensive, and has a certain latency because it has to call upon a remote control centre. Moreover, it is not secure because the communication between the antidiffusion grid and the control centre is not carried out by near field technology (it can be intercepted). There is no integrated device for warning the user in the case of inadequacy between the antidiffusion grid and the detector.

It is also known to use a system of Hall effect sensors and magnets for identifying the type of antidiffusion grid used. In such a system, the detector is equipped with two Hall effect sensors, and magnets are introduced in the grid support facing the Hall effect sensors. If the Hall effect sensors do not detect any magnet, the detector considers that there is no antidiffusion grid. The detector can identify up to three types of grid, by means of the two sensors, according to whether one of them or both of them detect a magnet. This type of grid data is recorded in the detector and in the image in the form of a code: 0, 1, 2, 3: 0 signifying “no grid”, and 1, 2, or 3 signifying a type 1, type 2 or type 3 grid respectively.

Such a system is limited because it can identify only three types of grid. There is no integrated device for warning the user in the case of inadequacy between the antidiffusion grid and the detector.

### SUMMARY OF THE INVENTION

One purpose of the invention is to overcome the abovementioned problems, and more particularly to be able to detect, in an autonomous, secure, simplified and fast (local) manner, a virtually unlimited number of antidiffusion grids and to determine if the grid to be used is compatible with the detector.

There is proposed, according to one aspect of the invention, a radiological imaging system comprising a portable X-ray image detector and an antidiffusion grid comprising a support for receiving the portable detector, wherein:
the portable detector is provided with a near distance radio communication module; and
the antidiffusion grid comprises a near distance radio communication tag, provided with a memory of at least 10 kilobytes comprising data representative of the antidiffusion grid, disposed in such a way as to be capable of communicating with the near distance radio communication module when the detector is fully inserted in the receiving support;
   the portable detector being configured for determining locally (i.e. without remote access) if the antidiffusion grid and the portable detector are compatible or incompatible from read data representative of the antidiffusion grid supplied to the near distance radio communication module by the tag.

Such a system makes it possible to detect, in an autonomous, secure, simplified and fast (local) manner, a virtually unlimited number of antidiffusion grids and to determine if the grid to be used is compatible with the detector.

The automatic identification of the antidiffusion grid, the verification of its compatibility with the detector and the integrated device warning the user make it possible to avoid an operational error likely to uselessly expose the patient to X-rays. The capability of the detector to carry to carry out the detection of the antidiffusion grid in an autonomous manner allows the use of this type of solution on radiology equipment which is not equipped with a control centre.

In one embodiment, the system comprises a module for blocking the functioning of the portable detector whilst declaring it as not ready for acquiring an image if the portable detector and the antidiffusion grid are incompatible.

Thus, the radiological imaging system is blocked when the antidiffusion grid is not compatible with the detector inserted in its support.

According to one embodiment, the system comprises a warning module configured to inform a user if the antidiffusion grid and the portable detector are compatible or incompatible.

Thus, the user is clearly informed of the compatibility or of the incompatibility of the portable detector and the antidiffusion grid.

In one embodiment, the warning module comprises:
a sound emitter configured for emitting a first sound signal in the case of compatibility of the antidiffusion grid and the portable detector, and for emitting a second sound signal in the case of incompatibility of the antidiffusion grid and the portable detector; and/or
a light emitter configured for emitting a first light signal in the case of compatibility of the antidiffusion grid and the portable detector, and for emitting a second light signal in the case of incompatibility of the antidiffusion grid and the portable detector.

According to one embodiment, the light emitter comprises light emitting diodes configured for flashing in green in the case of compatibility of the antidiffusion grid and the portable detector, and for flashing in red in the case of incompatibility of the antidiffusion grid and the portable detector.

The use of a such a conventional visual code is easily and immediately understandable.

According to one embodiment, the portable detector is configured for, during an image acquisition, applying image corrections corresponding to the type of the antidiffusion grid.

Thus, the processing of the acquired image is improved.

For example, the portable detector is configured for, during an image acquisition, inserting the data representative of the antidiffusion grid in the header of the computer file representing the acquired image.

Thus, in a fast, local and efficient manner, the characteristics of the antidiffusion grid used for acquiring the image is in the computer file representing the acquired image, and the system can then use these characteristics as input parameters of the image processing.

In one embodiment, the data representative of the antidiffusion grid comprise an identifier representative of the type of antidiffusion grid, and/or an identifier representative of the antidiffusion grid, and/or an identifier representative of its portrait or landscape orientation, and/or an identifier representative of the number of pairs of lines per centimetre, and/or an identifier representative of the form factor, and/or an identifier representative of the nominal focal length of the X-ray source, and/or an identifier representative of the minimum and maximum focal lengths of the X-ray source, and/or an identifier representative of the primary transmission rate of the X-rays of the device, and/or an identifier representative of the dimensions of the antidiffusion grid, and/or an identifier representative of the thickness of the absorbent material.

All of these data make it possible to accurately and locally identify the type of grid and its characteristics.

For example, the near distance radio communication module is of the near field communication NFC type, NFC being the acronym for “Near Field Communication” in English, and the near distance radio communication tag is an NFC tag.

The use of NFC communication is easy to implement and is inexpensive because many formats of tags, “tags” being the English term, allowing identification are available, and are generally inexpensive. Moreover, NFC communication is secure because it is carried out in in near field.

As a variant, the near distance radio communication module is of the radiofrequency identification RFID communication type, and the near distance radio communication tag is an RFID tag. This has the disadvantage of not being secure.

### BRIEF DESCRIPTION OF THE DRAWINGS

The invention will be better understood on examining several embodiments described as examples which are in no way limiting and which are illustrated by the appended drawings among which figures:

FIG. 1 is a diagrammatic illustration of a digital radiological imaging system, according to one aspect of the invention; and

FIG. 2 is a diagrammatic illustration of a portable X-ray image detector of a system as shown in FIG. 1, according to one aspect of the invention.

### DETAILED DESCRIPTION

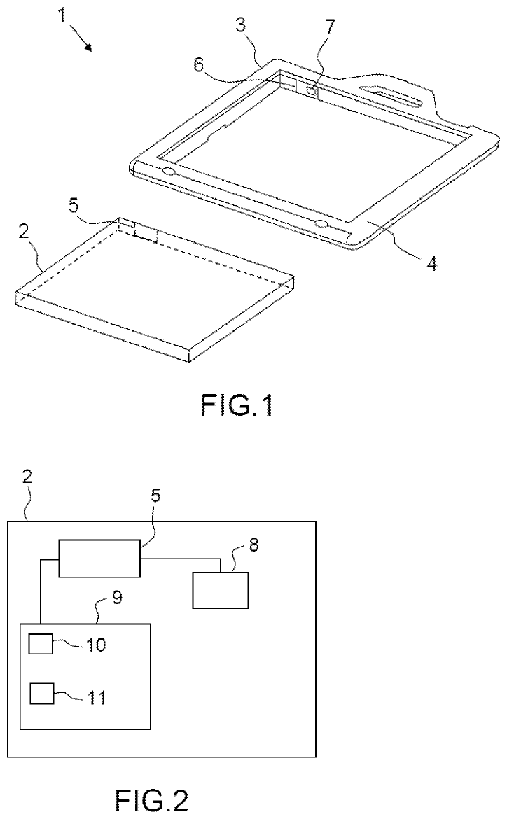

FIG. 1 is a diagrammatic representation of a radiological imaging system 1 according to one aspect of the invention.

A radiological imaging system 1 comprises a portable X-ray image detector 2 and an antidiffusion grid 3 comprising a support 4 for receiving the portable detector 2, wherein:
the portable detector 2 is provided with a near distance radio communication module 5; and
the antidiffusion grid 3 comprises a near distance radio communication tag 6, “tag” being the English term, provided with a memory 7 of at least 10 kilobytes comprising data representative of the antidiffusion grid 3, disposed such a way as to be capable de communicating with the near distance radio communication module 5 when the detector 2 is fully inserted in the receiving support 4.

The portable detector 2 is configured for locally determining (without connection to a remote server) if the antidiffusion grid 3 and the portable detector 2 are compatible or incompatible on the basis of the read data representative of the antidiffusion grid 3 supplied to the near distance radio communication module 5 by the tag 6.

FIG. 2 is a diagrammatic representation of a portable detector 2 according to one aspect of the invention, provided with a module 8 for blocking the functioning of the portable detector 2 whilst declaring it as not ready to acquire an image if the portable detector (2) and the antidiffusion grid (3) are incompatible.

Thus, there is no emission of X-rays when the portable detector 2 and the antidiffusion grid 3 are incompatible.

A warning module 9 can be configured for informing a user if the antidiffusion grid 3 and the portable detector 2 are compatible or incompatible.

The warning module 9 can comprise:
a sound emitter 10 configured to emit a first sound signal in the case of compatibility of the antidiffusion grid 3 and the portable detector 2, and to emit a second sound signal in the case of incompatibility of the antidiffusion grid 3 and the portable detector 2; and/or
a light emitter 11 configured to emit a first light signal in the case of compatibility of the antidiffusion grid 3 and the portable detector 2, and to emit a second light signal in the case of incompatibility of the antidiffusion grid 3 and the portable detector 2.

For example, the light emitter can comprise light emitting diodes configured to flash in green in the case of compatibility of the antidiffusion grid 3 and the portable detector 2, and to flash in red in the case of incompatibility of the antidiffusion grid 3 and the portable detector 2.

When the detector 2 is fully inserted in the support 4 of the antidiffusion grid 3, the near distance radio communication module 5 is facing the tag 6. If the antidiffusion grid 3 used is compatible with the portable detector 2, then the latter can emit a first particular sound corresponding to a compatibility and the light emitting diodes can flash in green.

On the contrary, if the antidiffusion grid 3 is not compatible with the portable detector 2, the latter can emit a second sound corresponding to an incompatibility and the light emitting diodes can flash in red.

The near distance radio communication module 5 then reads the data or information contained in the memory 7 of the tag, that is to say all of the characteristics of the antidiffusion grid 3 used.

The radiological X-ray imaging system 1 can then retrieve the data of the antidiffusion grid 3 by a software command sent to the X-ray detector and thus adjust the characteristics of the X-ray beam that it will generate for exposing the patient.

The portable detector 2 can be configured in order, during an image acquisition, to insert the data representative of the antidiffusion grid 3 in the header of the computer file representing the acquired image.

The X-ray detector 2 then writes all of the characteristics of the antidiffusion grid 3 used for acquiring an X-ray image in the header of the digital image (computer file representing the acquired image) that it has produced. The system then uses these characteristics as input parameters for its image processing.

This makes it possible to reduce the risk of errors in the data of the antidiffusion grid used for the acquisition and the processing of the image.

The data representative of the antidiffusion grid 3 can comprise an identifier representative of the type of antidiffusion grid, and/or an identifier representative of the antidiffusion grid, and/or an identifier representative its portrait or landscape orientation, and/or an identifier representative the number of pairs of lines per centimetre, and/or an identifier representative of the form factor, and/or an identifier representative of the nominal focal length of the X-ray source, and/or an identifier representative of the minimum and maximum focal lengths of the X-ray source, and/or an identifier representative of the primary transmission rate of the X-rays of the device, and/or an identifier representative of the dimensions of the antidiffusion grid, and/or an identifier representative of the thickness of the absorbent material.

The near distance radio communication module 5 can be of the NFC near field communication type and the near distance radio communication tag 6 is an NFC tag.

As a variant, the near distance radio communication module 5 can be of the RFID radio-identification communication type, and the near distance radio communication tag 6 is an RFID tag.

1. A radiological imaging system comprising a portable X-ray image detector and an antidiffusion grid comprising a support for receiving the portable detector, wherein:
the portable detector is provided with a near distance radio communication module; and
the antidiffusion grid comprises a near distance radio communication tag, provided with a memory of at least 10 kilobytes comprising data representative of the antidiffusion grid, disposed in such a way as to be capable of communicating with the near distance radio communication module when the detector is fully inserted in the receiving support;
the portable detector being configured for determining locally if the antidiffusion grid and the portable detector are compatible or incompatible from read data representative of the antidiffusion grid supplied to the near distance radio communication module by the tag.

2. The system according to claim 1, comprising a module for blocking the functioning of the portable detector whilst declaring it as not ready for acquiring an image if the portable detector and the antidiffusion grid are incompatible.

3. The system according to claim 1, comprising a warning module configured to inform a user if the antidiffusion grid and the portable detector are compatible or incompatible.

4. The system according to claim 3, wherein the warning module comprises:
a sound emitter configured for emitting a first sound signal in the case of compatibility of the antidiffusion grid and the portable detector, and for emitting a second sound signal in the case of incompatibility of the antidiffusion grid and the portable detector; and/or
a light emitter configured for emitting a first light signal in the case of compatibility of the antidiffusion grid and the portable detector, and for emitting a second light signal in the case of incompatibility of the antidiffusion grid and the portable detector.

5. The system according to claim 4, wherein the light emitter comprises light emitting diodes configured for flashing in green in the case of compatibility of the antidiffusion grid and the portable detector, and for flashing in red in the case of incompatibility of the antidiffusion grid and the portable detector.

6. The system according to claim 1, wherein the portable detector is configured for, during an image acquisition, applying image corrections corresponding to the type of the antidiffusion grid.

7. The system according to claim 6, wherein the portable detector is configured for, during an image acquisition, inserting the data representative of the antidiffusion grid in the header of the computer file representing the acquired image.

8. The system according to claim 1, wherein the data representative of the antidiffusion grid comprise an identifier representative of the type of antidiffusion grid, and/or an identifier representative of the antidiffusion grid, and/or an identifier representative of its portrait or landscape orientation, and/or an identifier representative of the number of pairs of lines per centimetre, and/or an identifier representative of the form factor, and/or an identifier representative of the nominal focal length of the X-ray source, and/or an identifier representative of the minimum and maximum focal lengths of the X-ray source, and/or an identifier representative of the primary transmission rate of the X-rays of the device, and/or an identifier representative of the dimensions of the antidiffusion grid, and/or an identifier representative of the thickness of the absorbent material.

9. The system according to claim 1, wherein the near distance radio communication module is of the near field communication NFC type, and the near distance radio communication tag is an NFC tag.

10. The system according to claim 1, wherein the near distance radio communication module is of the radiofrequency identification RFID communication type, and the near distance radio communication tag is an RFID tag.
